(19) United States
(12) Patent Application Publication
(10) Pub. No.: US 2021/0121639 A1
(43) Pub. Date: Apr. 29, 2021

(54) HANDHELD PRINTER FOR CONTROLLED MIXING AND DELIVERY OF MULTI-COMPONENT POLYMERS/BIOMATERIALS

(71) Applicant: Rowan University, Glassboro, NJ (US)

(72) Inventors: Amir K. Miri Ramsheh, Haddonfield, NJ (US); Matthew William Malpica, Glassboro, NJ (US)

(21) Appl. No.: 17/073,506

(22) Filed: Oct. 19, 2020

Related U.S. Application Data
(60) Provisional application No. 62/926,906, filed on Oct. 28, 2019.
(60) Provisional application No. 63/000,194, filed on Mar. 26, 2020.

(51) Int. Cl. A61M 5/315; B29C 48/02; A61M 5/31

(52) U.S. Cl. CPC A61M 5/31596; A61M 5/3129; B29C 48/02

(57) ABSTRACT

Compact, light-weight, handheld printer devices are capable of delivering multi-part materials in mixed form, with controllable dwell-time within the device to allow for a desired amount of physical mixing and chemical cross-linking, while also allowing for deposition of the material directly onto a deposition site. A pen-style printer having a slim but elongated form factor is particularly suitable for depositing biomaterials directly onto a surgical site in the throat. The device may be configured to receive and dispense materials directly from conventional syringes. The printer device may be configured for dispensing and mixing materials having two or more component parts, and may include a leadscrew mechanism providing mechanical advantage, for dispensing higher-viscosity materials, and/or an enhanced degree of precision in dispensing a quantity of material. The mixing time and quantities can be controlled by varying one or more of syringe size and/or injector passage size and mixing chamber length.

### CROSS-REFERENCE TO RELATED APPLICATION

This application claims the benefit of priority, under 35 USC 119(e), of U.S. Provisional Patent Application Nos. 62/926,906 and 63/000,194, filed Oct. 28, 2019, and Mar. 26, 2020, respectively, the entire disclosures of both of which are hereby incorporated herein by reference.

### FIELD OF THE INVENTION

The present invention relates generally to a device for use with polymer-based sealants, in polymer injections, in surgical procedures, and more particularly, as a printer device for use in surgical procedures to deliver biomaterials directly to surgical tissue sites, as may be required in voice-and spinal-related surgeries, or in other applications to combine and deposit, in controlled fashion, multi-part materials.

### DISCUSSION OF RELATED ART

It is estimated that up to 10% of the general population has some type of voice abnormality during their life causing them to lose work or change professions. Depending on the severity, disorders of the larynx require different treatment approaches, such as voice therapy, laryngeal surgery, and vocal fold (VF) augmentation. VF augmentation is a surgical procedure involving a VF injection that delivers a biomaterial to the VF tissue. This procedure is routinely performed to treat a variety of laryngeal disorders, including unilateral paralysis, paresis, atrophy, scar, and sulcus vocalis.

Low back pain caused by intervertebral disc degeneration affect 90% of US adults at some point in their lives. The most common surgical treatments for disc degeneration are spinal fusion and total disc arthroplasty, both of which are highly invasive surgical procedures requiring long recovery periods. Biomaterials have been developed as an alternative treatment option in which polymer implants are injected into the nucleus of the disc non-invasively through a small gauge needle, hardening in situ into a permanent implant that restores the mechanics of the spine. Most of current developments lack the control over deposited biomaterials and suffer from misplacement of the injected biomaterial.

Multiple-part curable resins such as epoxies are commonly mixed using a multiple-part syringe equipped with an exit nozzle. The materials contained in the syringe are dispensed and mixed by depressing the syringe plunger, thereby forcing the resin components from the syringe barrels into a mixing element (where the resin parts are intermixed with one another) and out the exit nozzle. Similar apparatuses have been known in which fluids to be mixed have been dispensed by double-barreled syringe or caulking gun type dispensers (U.S. Pat. Nos. 3,309,814, 4,041,463, and 4,538,920, 6,079,868A). Most of such cases require preparation and they lack control over deposition and mixing time.

Motivated by regenerative medicine strategies, numerous efforts have been made to repair and restore tissue in injured VFs/skin using biomaterials. Presently available VF/skin biomaterials, which are variable in their long-term duration, require significant preparation efforts and require post-surgery treatments. Current biomaterial interventions lack proper mechanical stability and biomaterial-tissue adhesion thus fail to restore native tissue, and cannot restore the biophysical function. As a result, VF/skin augmentation clinical outcomes are inconsistent, and no biomaterial-based intervention exists that can adequately restore native tissue.

To date, most studies have focused on selective application of an individual therapeutic methodology in the design of biomaterials. The main limitations of these biomaterial approaches are inadequate stiffness and low adhesion to host tissue. It is crucial to achieve steady adhesion to surrounding tissue without limiting the desired function of the tissue. Current injections from a needle are also inadequate for large voids, such as surgically-resected tumor regions. An ideal implant should adhere and seal in situ to prevent dislodgement and ingestion into other organs. To overcome these limitations, highly controlled deposition of tissue-adhesive and tunable bioinks using novel 3D printing techniques can enable rebuilding of the resected portion of host tissue. In case of non-medical applications, polymer-based sealers and glues require preparation before using them in a gun. The control over the delivery of polymeric materials is normally limited because of the size of heavy handling used for pushing materials.

What is needed is a printer device capable of delivering advanced, multi-part materials, such as certain biomaterials and polymer-based compositions, in mixed form, with controllable dwell-time to allow for proper cross-linking, etc., while also allowing for deposition of the well-mixed material in a controlled fashion, e.g., directly to a tissue site to be repaired, with precision, for example, so a biomaterial implant can adhere and seal in situ to prevent dislodgement and aspiration. Further, what is needed is a customizable handheld pen-style printer, which allows for the in situ deposition of self-healing and polymer based hydrogels, which can be used to fabricate stable and functional VF/skin/spinal implants. Such hydrogels work based on guest-host (or Part A-Part B) physical interactions of a macrocyclic host and a complementary guest molecule. UV crosslinking of methacrylate groups may be used in our materials for long-term stability of selected implant. In addition, dispensing devices of this kind are useful in the application of a variety of pasty or highly-viscous products such as adhesives, joint filler agents, foams, sealants, molding compounds and others, for industrial or other non-medical purposes. Such products typically consist of two or more components which are stored separately and mixed at the time of use in order to start a chemical reaction between them, usually causing a solidification or hardening of the resultant mass.

### SUMMARY

The present invention relates to printer devices that are capable of delivering single-part or multi-part materials (e.g., biomaterials and polymers) in mixed form, with controllable dwell-time within the device to allow for a desired amount of physical mixing, chemical cross-linking, etc. while also allowing for deposition of the mixed multi-part material directly onto an intended deposition site, e.g., using a generally compact, light-weight, manually-operated implement allowing the operator to deposit material manually and with precision, according to the user's manual dexterity.

In accordance with one aspect of the present invention, the present provides a printer device suitable for depositing biomaterials onto biological tissue. An exemplary printer device has a pen-style form factor and is generally well-suited to deposition of biomaterials onto biological materials in the neck region of the body, e.g., on vocal fold (VF) tissue. In accordance with the present invention, a printer device is provided that is capable of delivering advanced biomaterials in mixed form. Common current biomaterials include mixable two-part hydrogels and/or other materials that are not terribly viscous, and are readily dispensed by hand from a syringe-link device.

In accordance with another aspect of the present invention, the present provides a printer device suitable for depositing materials having more than 2 component parts. In such embodiments, the device may be structured for receiving multiple syringes, etc., and for dispensing and mixing those materials in similar fashion.

In accordance with yet another aspect of the present invention, the present provides a printer device suitable for depositing materials having two or more component parts with relatively higher viscosities (such as many polymers used for industrial purposes) and/or where higher dispensing precision is required. In such embodiments, the device includes a leadscrew mechanism operable to mechanically drive the plunger using mechanical advantage. The leadscrew mechanism may include a lever mounted to a lever bevel gear such that turning the lever about an axis of rotation causes the lever bevel gear to rotate, and to thereby cause corresponding longitudinal motion of the plunger that it impinges upon due to mating threads of the bevel lead screw and the printer device.

### BRIEF DESCRIPTION OF THE FIGURES

An understanding of the following description will be facilitated by reference to the attached drawings, in which:

FIG. 1 is an image of damaged vocal fold tissue;

FIG. 2 is an illustration of a laryngoscopy procedure that may be used to visualize the vocal fold tissue of FIG. 1;

FIG. 3 is a side view of a pen-style printer device for printing biomaterials in accordance with an exemplary embodiment of the present invention;

FIG. 4 is a perspective view of the printer device of FIG. 3;

FIG. 5 is another perspective view of the printer device of FIG. 3;

FIG. 6 is a partial enlarged side view of the printer device of FIG. 3;

FIG. 7 is a rear perspective view of the printer device of FIG. 3;

FIG. 8 is a partial enlarged perspective view of the syringe holder and plunger of the printer device of FIG. 3;

FIG. 9 is a bottom view of the printer device of FIG. 3; and

FIG. 10 is a partial view of an exemplary coaxial injector for the printer device of FIG. 3;

FIG. 11 is a partial view showing an alternative embodiment of a coaxial injector for the printer device of FIG. 3; and

FIG. 12 is a front view of an alternative printer device in accordance with an alternative embodiment of the present invention;

FIG. 13 is a rear view of the printer device of FIG. 11;

FIG. 14 is a rear view of the printer device of FIG. 11, shown with portions removed for illustrative clarity;

FIG. 15 is a perspective view of the bevel lead screw of the printer device of FIG. 11;

FIG. 16 is a perspective view of the lever bevel gear of the printer device of FIG. 11;

FIG. 17 is a perspective view of another alternative printer device in accordance with another exemplary embodiment of the present invention;

FIG. 18 is a side view of an exemplary coaxial injector for the printer device of FIG. 17;

FIG. 19 is a partial view of an exemplary coaxial injector for the printer device of FIG. 17;

FIG. 20 is a partial view of an exemplary coaxial injector for the printer device of FIG. 17; and

FIG. 21 is a perspective view of another alternative printer device in accordance with another exemplary embodiment of the present invention.

### DETAILED DESCRIPTION

The present invention relates to printer devices that are capable of delivering multi-part materials (e.g., biomaterials and polymers) in mixed form, with controllable dwell-time within the device to allow for a desired amount of physical mixing, chemical cross-linking, etc. while also allowing for deposition of the mixed multi-part material directly onto an intended deposition site, e.g., using a generally compact, light-weight, manually-operated implement allowing the operator to deposit material manually and with precision, according to the user's manual dexterity.

In accordance with one aspect of the present invention, the present provides a printer device for depositing biomaterials onto biological tissue. The exemplary printer device has a pen-style form factor and is generally well-suited to deposition of biomaterials onto biological materials in the neck region of the body, e.g., on vocal fold (VF) tissue. In accordance with the present invention, a printer device is provided that is capable of delivering advanced, two-part, biomaterials in mixed form, with controllable dwell-time within the device to allow for a desired amount of cross-linking, while also allowing for deposition of the biomaterials directly onto the tissue site to be repaired, with precision, so the biomaterial implant can adhere and seal in situ to prevent dislodgement and aspiration.

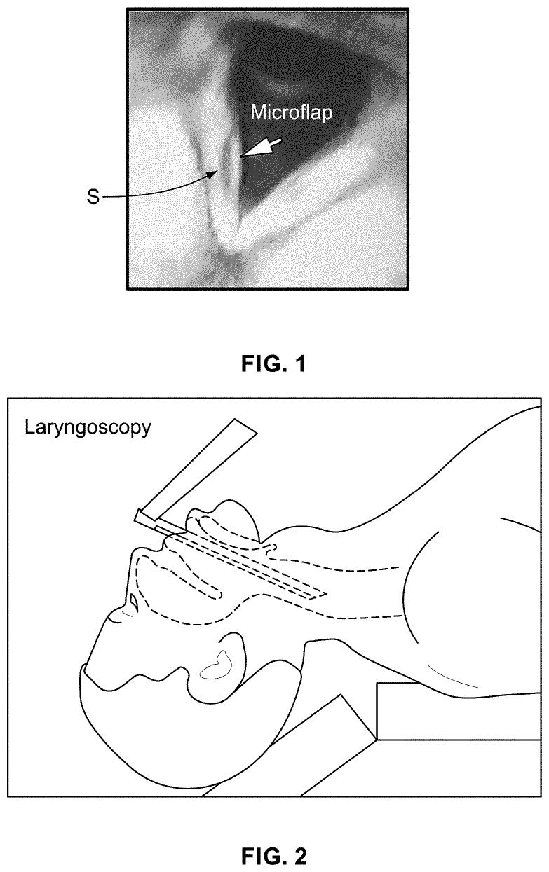

An exemplary embodiment of the present invention is discussed below for illustrative purposes. Referring now to FIG. 1, an image of damaged VF tissue, having a scarred portion S, is shown. Such VF tissue may be visualized in a conventional laryngoscopy procedure, illustrated in FIG. 2, as known in the prior art. The damaged vocal tissue is of a type that would be repaired in a VF repair surgical procedure.

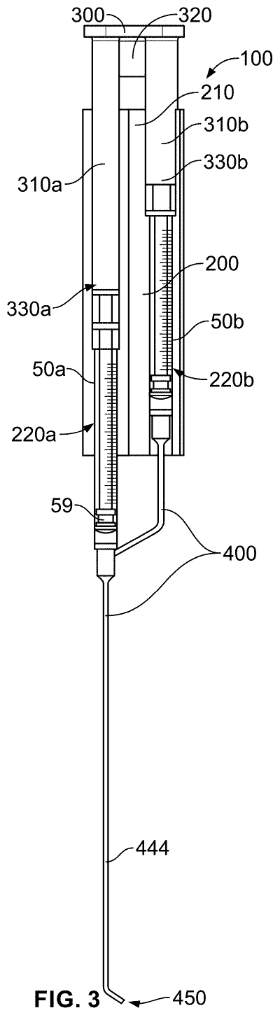

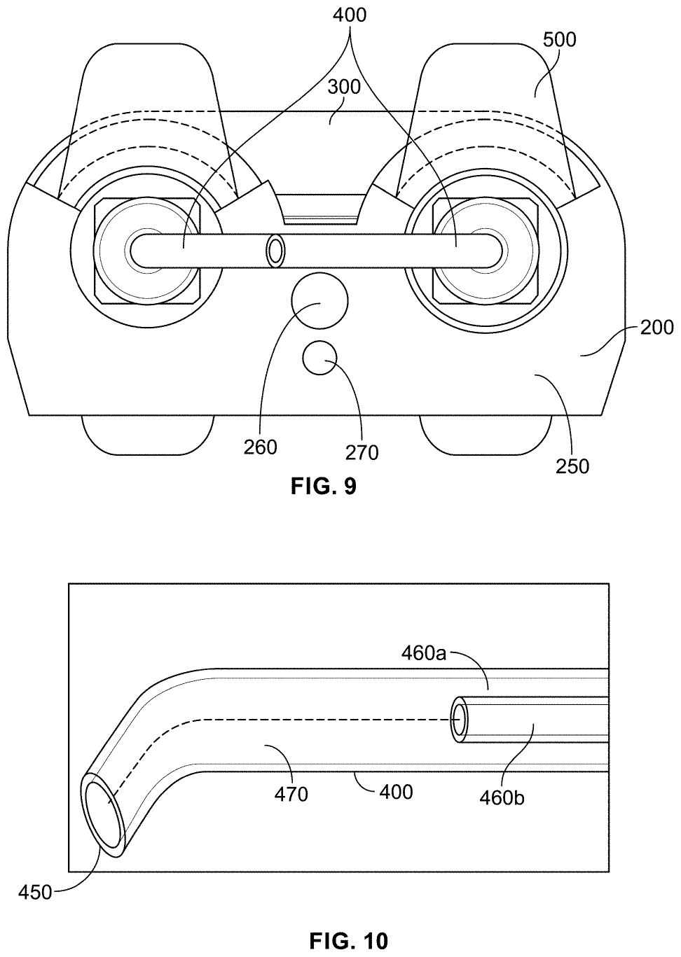

FIGS. 3-9 show an exemplary printer device 100 in accordance with an exemplary embodiment of the present invention. Referring now to FIGS. 3-9, the exemplary printer 100 includes a syringe holder 200, a plunger 300, and an injector 400. The syringe holder 200 is configured to hold two (or more) conventional syringes 50 (two for the exemplary holder 200 shown in FIG. 3) containing biomaterial components of a two-part biomaterial intended to be delivered to the surgical site. As will be appreciated by those skilled in the art, biomaterials are often sold or distributed in relatively small, e.g., 1 mL, 3 mL or 5 mL, conventional syringes. This range of volume would be enough for most of VF augmentation treatments. As will be further appreciated by those skilled in the art, a respective conventional syringe 50 typically includes a barrel 52 having a barrel flange 53 at one end and a Luer lock/tip/needle adapter/fitting 55 at its outlet, and a plunger body 56 having a plunger flange 57 at one end and a fluid-tight seal 59 at the other, as is conventional, and as best shown in FIG. 3.

Each of the two or more syringes 50a, 50b, may contain a respective one of a multipart (e.g., multiphase) material (such as a biomaterial). For example, it may be desirable to mix biomaterial component A contained in syringe 50a with biomaterial component B contained in syringe 50b to produce a composite biomaterial to be delivered to a surgical site via the printed 100. The exemplary syringe holder 200 is configured for holding two syringes of a two-part composite biomaterial, but it will be appreciated that the syringe holder 200 may be configured to hold any number of syringes of a multipart biomaterial composites in accordance with the present invention.

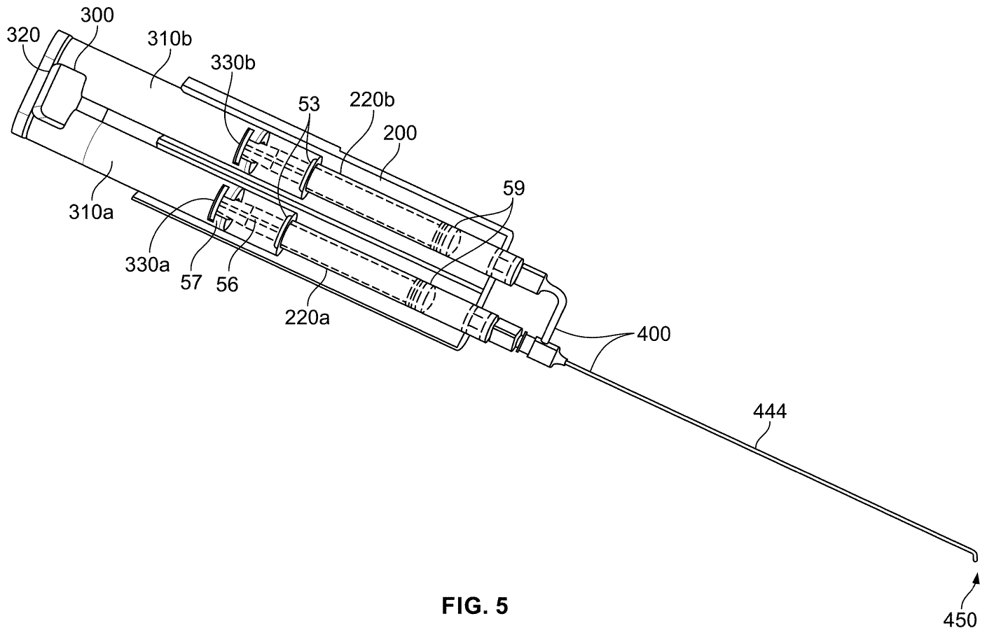

More particularly, the syringe holder 200 comprises a holder body 210 defining one or more, and preferably two or more, individual channels 220a, 220b, as best shown in FIG. 5. The channels extend along respective and distinct axes. In one embodiment, the channels extend along parallel axes.

Each channel 220a, 220b is dimensioned to receive a respective conventional syringe 50a, 50b. Further, the syringe holder 200 is configured to restrain each syringe 50a, 50b against longitudinal motion within the channel, in at least one direction, e.g., to restrain the barrel while the plunger is being advanced relative to the barrel. For example, each channel 220a, 220b may define a socket 222 for receiving a portion of a respective syringe's barrel flange 53, as best shown in FIGS. 3, 6 and 8. Alternatively, or additionally, the syringe holder 200 may be provided with a shoulder/stop 224 for abutting a portion of the barrel, e.g., near the barrel flange 53 and/or near the Luer lock 55, as best shown in FIG. 5.

The channels 220a, 220b may be configured to hold syringes/barrels of the same size (e.g., two 3 mL syringes) or of different sizes (e.g., a 3 mL syringe in one channel and a 5 mL syringe in another channel). Preferably, each channel 220a, 220b is dimensioned to hold one of a 1 mL, a 3 mL, a 5 mL or a 10 mL syringe. The sizes of the syringes and barrels may be selected to correspond to desired mixing ratios of the biomaterial components contained in the individual syringes. For example, for a 50:50 mixture of two components, it may be desirable to use syringes of the same volume/barrel size and channels of the same size, so that equal volumes are dispensed from each syringe in response to equal advancement of their plungers. For other than 50:50 mixtures, it may be desirable to use syringes of different volumes so that different volumes of each biomaterial component may be dispensed from each syringe in response to equal advancement of their plungers. More particularly, the use of syringes of different sizes (volumes) allows for inequal mixing ratios with pre-defined proportions of materials. The present invention also provides a multiple-plunger holder with flexible control over any of the syringes. Accordingly, it should be noted that the present invention also contemplates an arrangement involving inequal advancement of plungers to obtain both equal and inequal mixtures of the components of various syringe.

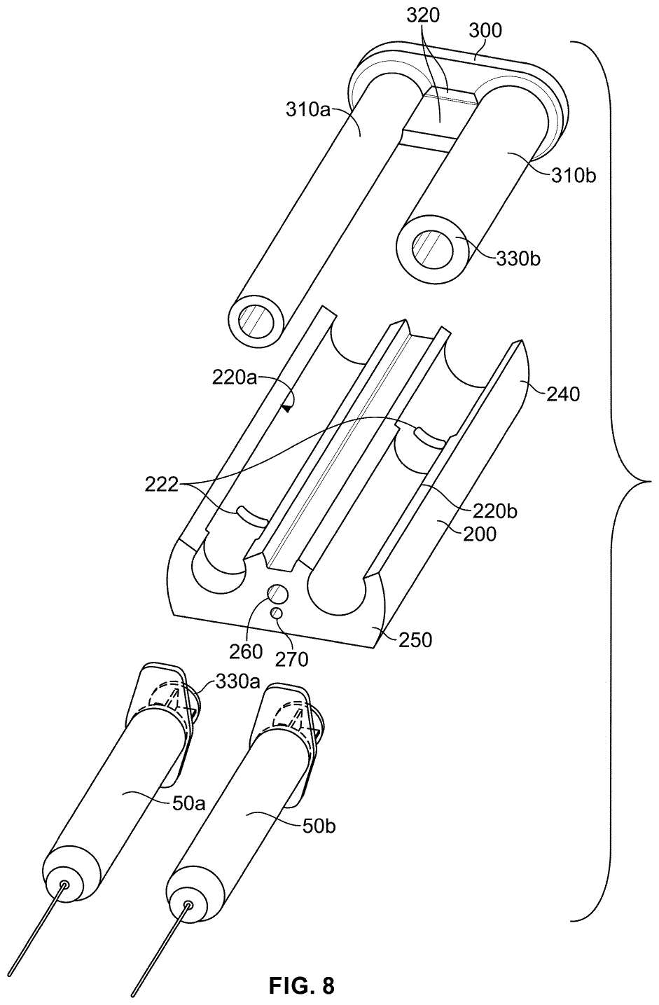

In the exemplary embodiment, the syringe holder 200 further defines at least one through-bore extending and open to its proximal end 240 and its distal end 250. In the exemplary embodiment shown, the syringe holder 300 defines a first through-bore 260 dimensioned to admit passage of an endoscope, and a second through-bore 270 dimensioned to admit passage of a light source, as best shown in FIGS. 7-9. For example, a through-bore having a diameter of about 4-5 mm may be suitable for admitting passage of the endoscope, as many endoscopes have an external diameter or about 3.5 mm. By way of further example, a through-bore having a diameter of about 1-2 mm may be suitable for admitting passage of a fiber optic light source.

The device's plunger 300 is configured to have one or more bosses 310a, 310b for abutting the plunger flanges 57 of the syringes 50a, 50b. In this embodiment, the bosses are connected by a common base 320 to form an integral unit, to cause synchronized advancement of the bosses 310a, 310b. In some embodiments, the channels may be parallel, in which cases the bosses extend in parallel fashion. In other embodiments, the bosses may not be joined, and may not be part of an integral unit, so they may be advanced asynchronously.

In some embodiments, two or more syringes may be aligned longitudinally within the syringe holder 200. In such an embodiment, the ends 330a, 330b of the bosses 310a, 310b may be longitudinally aligned. In the embodiment shown in FIGS. 3-9, syringes 50a, 50b are misaligned longitudinally in the holder 200, and correspondingly, the ends 330a, 330b of the bosses 310a, 310b are correspondingly misaligned, as best shown in FIG. 7.

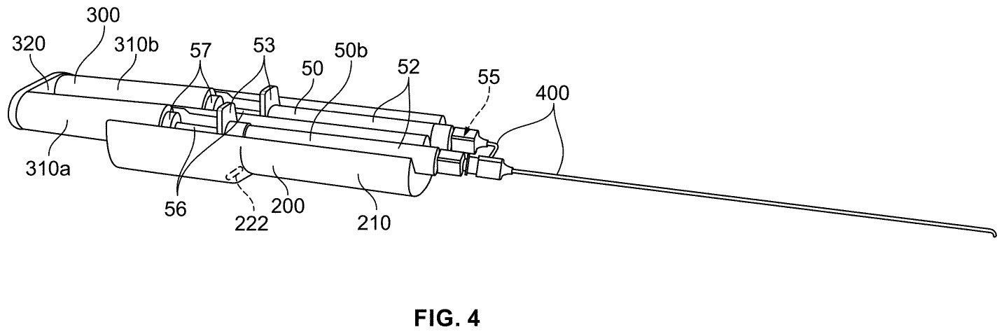

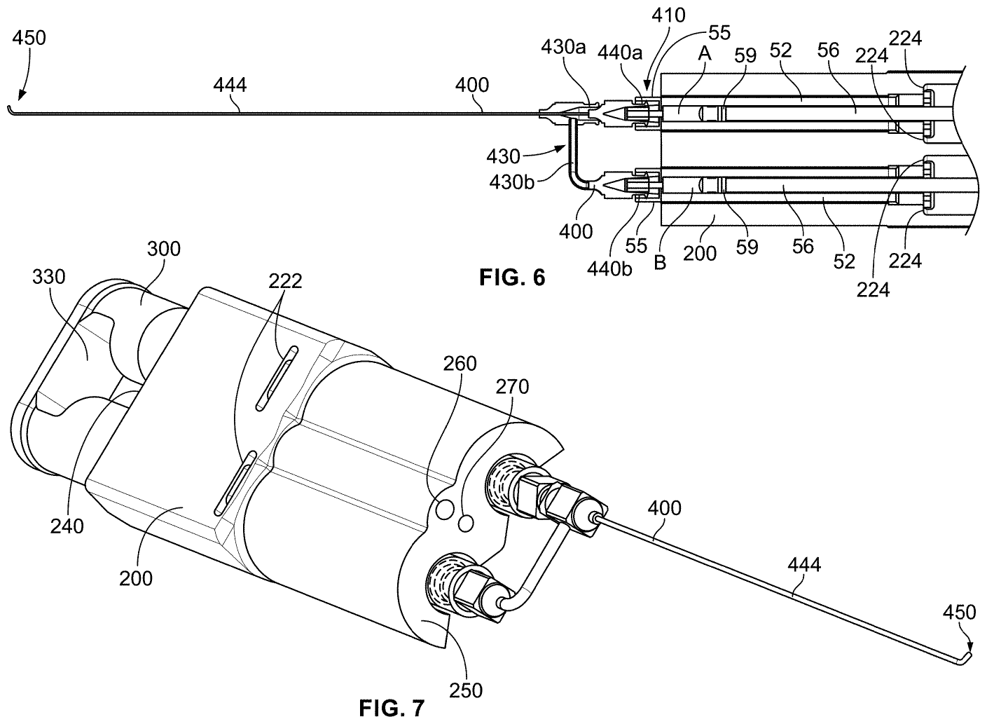

The device's injector 400 has a proximal end 410 and a distal end 420, as shown in FIG. 6. The injector 400 includes a branched portion 430 near its proximal end, and a coextending portion 444 terminating in an open tip portion 450. The branched portion 430 defines separate dedicated conduits 430a, 430b, each terminating in a connector 440a, 440b complementary to a fitting/connector 55 on the syringes, e.g., a Luer-lock style connector, as best shown in FIG. 6. In some embodiments, the connectors 55 at the distal ends of the syringes 50a, 50b may be longitudinally aligned, and thus the connectors of the dedicated conduits 430a, 430 may be aligned, as shown in FIGS. 4-9. In other embodiments, the syringes' connectors 50 are misaligned, and correspondingly, the connectors of the dedicated conduits 430a, 430 are correspondingly misaligned, as shown in FIG. 3. Accordingly, each conduit's connector may be connected to a respective connector of a respective syringe 50a, 50b, such that material passed from each syringe 50a, 50b travels through a respective dedicated conduit 430a, 430b.

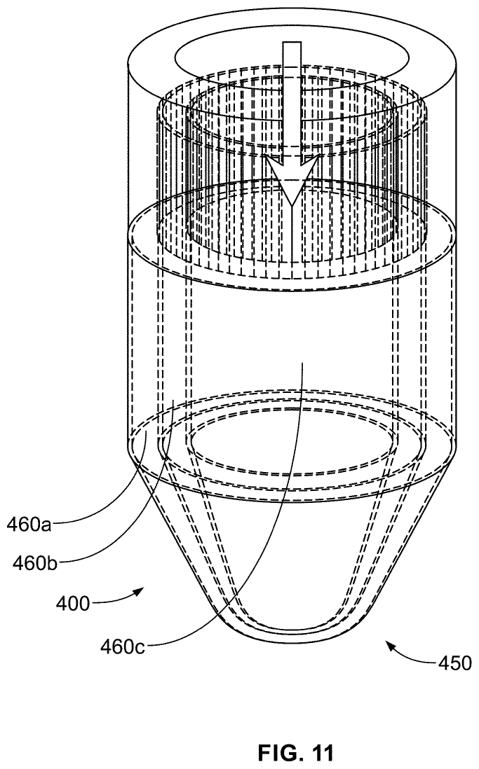

The injector's coextending portion 444 is configured to have at least one, and preferably at least two, distinct internal passages 460a, 460b extending along a common axis, e.g., side-by-side, so that separate component materials can be passed separately through at least a portion of the injector's length, as best shown in FIGS. 10 and 11. In a preferred embodiment, at least a portion of the passages are coaxial, as shown in FIGS. 10 and 11. The injector, and particularly the coextending portion, is preferably elongated, such that the tip portion is disposed approximately 15 cm or more, and preferably about 17 cm, from the distal end of the syringe holder 200, as this length is advantageous for allowing the tip 450 to reach likely surgical sites within the neck while the syringe block is maintained near the patient's open mouth.

FIG. 10 is a partial view of an exemplary injector 400 defining coaxial passages 460a, 460b that are not fully coextensive, and thus do not extend individually all the way to the distal tip/nozzle of the tip portion 450. Accordingly, in this embodiment, the two component materials A, B travel separately and do not mix within a portion of coextending portion 444 of the injector 400, and rather are kept separate until they reach a common, mixing portion 470 of the coextending portion 444 of the injector 400. In the mixing portion 470, material components A and B mix and flow together within the injector 400. Accordingly, for cross-linkable biomaterials for example, cross-linking may occur in the mixing region 470, before exiting at the distal end of the tip portion 450 of the injector 400, and before being deposited onto the patient's bodily tissue. It will be appreciated that such an injector may be structure to provide any desired number of passages.

It should be appreciated that the sizes and relative sizes of the respective passages of the injector, and/or the overall size of the injector, can be varied, and matched to the volumes of the syringes and/or desired volumes of material components/biomaterials desired to be delivered.

FIG. 11 is a partial view of an alternative exemplary injector 400 defining three coaxial passages 460a, 460b, 460c that are fully coextensive, and extend to the distal tip/nozzle of the tip portion 450. Accordingly, in this type of embodiment, the component materials A, B, C do not mix within the injector 400, and rather are kept separate until deposition, as they are co-extruded from and exit the tip portion 450 of the injector 400. It will be appreciated that such an injector may be structure to provide any desired number of passages.

It should be noted that the extent of cross-linking prior to deposition onto the patient's bodily tissue can be controlled by varying the flow rate of the materials via the printer device/injector. For example, this may be done manually by control of the advancement of the plunger 300. Alternatively, this may be done in automated fashion, e.g., using a mechanically driven mechanism, e.g., using a motor-driven screw drive, to advance the plunger 300 (or separate portions of the plunger, corresponding to each syringe) mechanically to reliability provide a desired flow rate, and a desired dwell time in the printer 100, e.g., to allow for a desired level of cross-linking within the printer prior to deposition of the material onto the patient's bodily tissue or other deposition site.

Further, the extent of cross-linking prior to deposition can be controlled by varying the structure of the injector. For example, the coextending portion 444 may be structure to have a longer or shorter mixing portion 470, to cause greater or lesser mixing, and to provide greater or lesser dwell time allowing for cross-linking for a given flow rate, as may be desired for the materials to be used.

The syringe holder 200 and plunger 300 may be constructed of a plastic material, and may be configured for single-use or sterilization and reuse. The injector 400 may be constructed of any suitable materials, but is preferably constructed of stainless steel or another metal for easy sterilization and reuse, as will be appreciated by those skilled in the art.

In use, syringes 50a, 50b loaded with desired materials (e.g., biomaterial components). The connectors 440a, 440b, of the injector 400 may then be mated to the complementary connectors/fittings 55 of both syringes 50a and 50b.

The syringes 50a and 50b may be loaded into the channels 220a, 220b of the syringe holder 200, and may be positioned to register with any sockets 222 or shoulders/stops 224 for longitudinally restraining the syringes within the holder 200. The plunger 300 may then be aligned with the channels 210a, 210b of the syringe holder 200, with the bosses 310a, 310b protruding in an arrangement corresponding to any axial misalignment of the syringes 50a, 50b, and be advanced into the syringe holder 200 until the ends 330a, 330b of the bosses 310a, 310b, abut both respective plunger flanges 57 of the syringes 50a, 50b.

The printer device 100 is then fully assembled and may be used as desired. For example, the printer 100 may then be advanced into the patient's mouth and throat (e.g., using a standard Hollinger or other laryngoscope or support-free setup), feeding the injector down the throat, and advancing the tip toward the vocal fold or other surgical site. As part of this process, a light source may be advanced through a first through-bore 270, and an endoscope may be advanced through a second through-bore 260, of the syringe holder 200 to provide illumination and visualization of the surgical site. When used in this manner, the laryngoscope and/or the light source serve to support and stabilize the printer during use, which can be advantageous.

When the tip 250 of the injector 400 is positioned at the surgical/deposition site, the plunger 300 may be advanced. As the plunger 300 is advanced, it correspondingly advances the individual plungers 56 of the individual syringes 50a, 50b. This causes component materials A, B to exit the respective syringes 50a, 50b, to pass through the injector 400, and to mix in the mixing chamber 270, if provided. Due to coordination of the materials, syringe volumes, flow rates, and injector/mixing chamber configuration, suitably cross-linked material (either uncross-linked, partially cross-linked or fully cross-linked, as desired) will exit via the tip 450 of the injector 400 and be deposited directly onto the bodily tissue at the surgical site or other deposition area.

Accordingly, the printer may be used to provide controlled delivery of self-healing, click-chemistry based, and shear-thinning biomaterials/hydrogels to VF tissue in voice surgery. Further, the printer may be used to mix, within the printer, self-healing and click-chemistry based hydrogels before reaching the surgical site tissue, and/or to deliver composite hydrogels and/or cells.

In certain embodiments, photo-crosslinkable hydrogels maybe used as the biomaterials, and a light source for photo-crosslinking the component materials may be passed though the through-bore 270 of the syringe holder to crosslink deposited materials in situ, after deposition onto the bodily tissue of the patient.

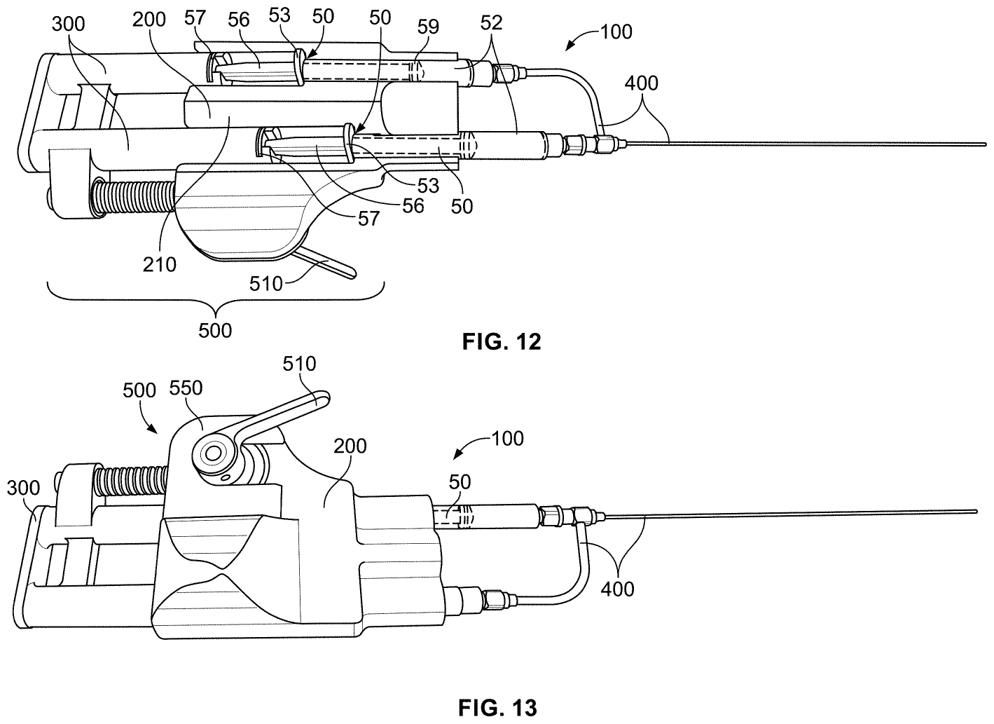

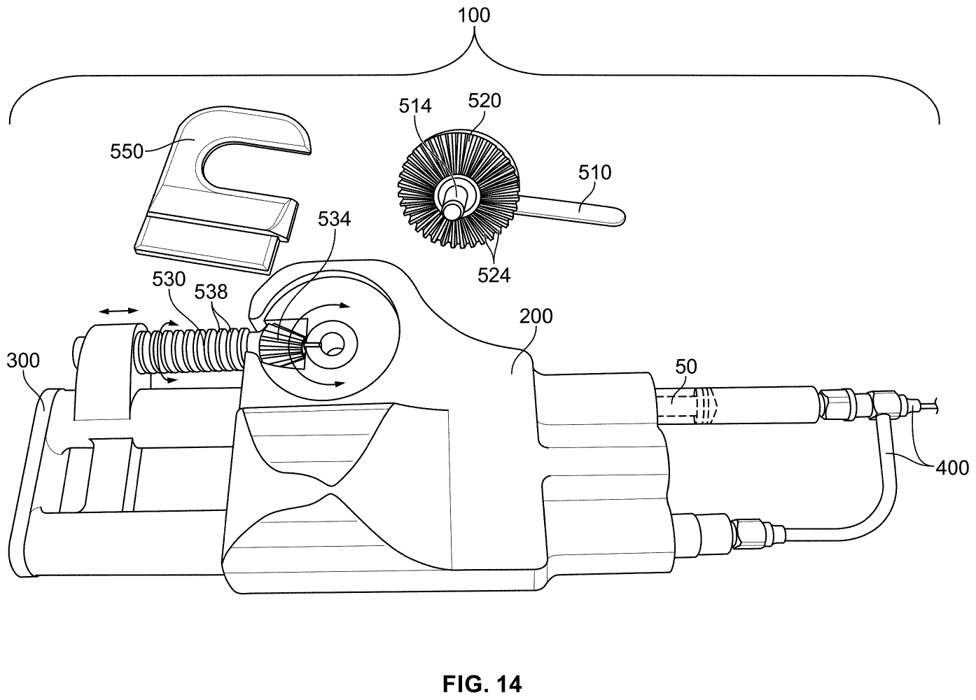

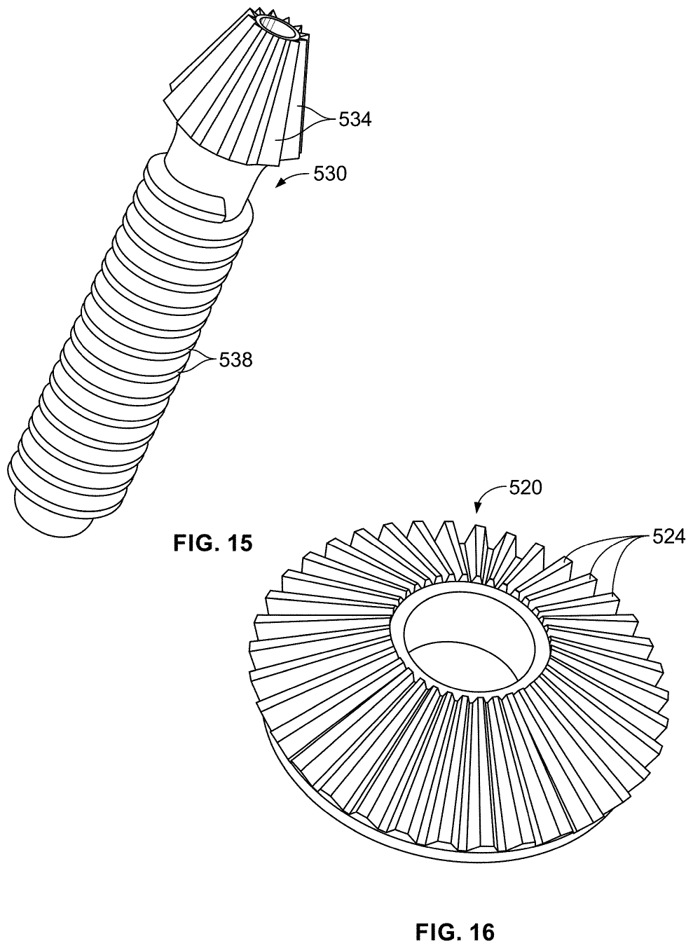

Referring now to FIGS. 12-16, an alternative embodiment of a printer device is shown. This printer embodiment is similar to the printer embodiment described above, except that it further includes a leadscrew mechanism 500 operable to mechanically drive the plunger using mechanical advantage, which may be helpful particularly for relatively more viscous materials or where enhanced control of the amount of material dispensed is desired. As best shown in FIG. 14, this printer device 100 includes a lever 510 mounted to a lever bevel gear 520. Turning the lever 510 about an axis of rotation causes the lever bevel gear to rotate about the axis. Teeth 524 of the lever bevel gear 520 mesh with complementary teeth 534 of a bevel lead screw 530, which is corresponding caused to rotate by rotation of the lever bevel gear 520, and to cause longitudinal motion of the plunger 300 that in impinges upon due to mating threads 538 of the bevel lead screw 530 and the plunger 300 of the printer device 100, as will be appreciated from FIGS. 12-16. In this example, a lever cover 550 slots into the main holder and over the flange of the lever to keep the lever 510 and level bevel gear 520 contained in the system and keep the lever bevel gear 520 and bevel lead screw 530 is a meshed arrangement with their gear teeth in contact. The cover 550 may be constructed to be easily removable so the drive mechanism can be accessed easily to enable planned future modularity of the drive mechanism by the user.

More particularly, in this exemplary embodiment, the lever 510 is attached to a pin 514 inside the bevel gear 520 to transmit torque via a one-way needle bearing/contact when moving against the free spinning direction, in accordance with conventional constructions well-known in the art. Accordingly, the needle bearing is used around the pin to create a ratchet type motion, so that only a single direction of the lever is operable to drive the plunger. Thus, the lever 510 provides mechanical advantage when turning the bevel gear. A torsion or other spring may be provided to reserve motion energy and restore the lever to a default position and/or provide rotational limits to force movement to be within a range (ex. 40°). Such a spring can be added between the lever 510 and cover 550.

The lever bevel gear interconnects the lever and the bevel lead screw. In one embodiment, a 40° rotation of the bevel gear will move the plunger 1 mm with corresponding ratio and pitch, but these can be varied as desired. The bevel lead screw transforms the rotational motion input into a vertical/longitudinal plunger motion. An exemplary gear ratio is 3:1 with a screw pitch of 3 mm.

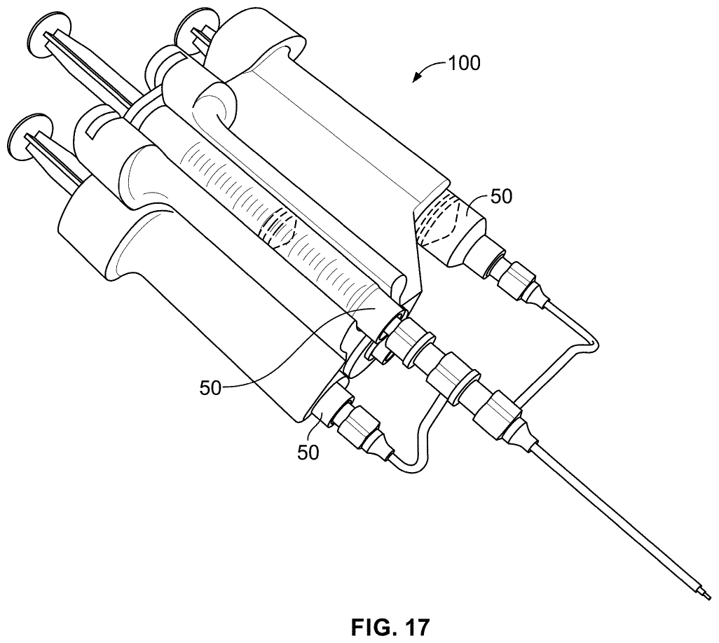

It should be noted that in some applications (such as applications in the construction, engineering and/or automotive industries), including applications other than printing of biomaterials, multipart/multiphase polymers or other materials are desired to be used that require mixing of more than two component materials. Those component materials may require concurrent mixing or materials, or sequential mixing of two or more of the component materials wither other component materials. Each may require a different dwell/mixing time to allow for proper cross-linking, etc. FIGS. 17-20 illustrate another exemplary embodiment that is illustrative of a printer device capable of delivery advanced, multi-part materials in mixed form, with controllable dwell-time to allow for proper cross-linking, using three or more component materials, and three or more syringes.

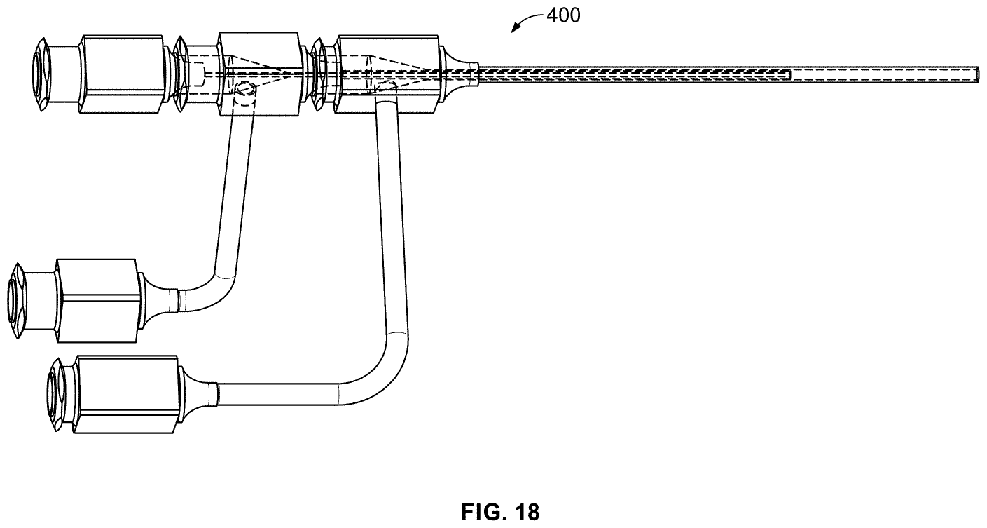

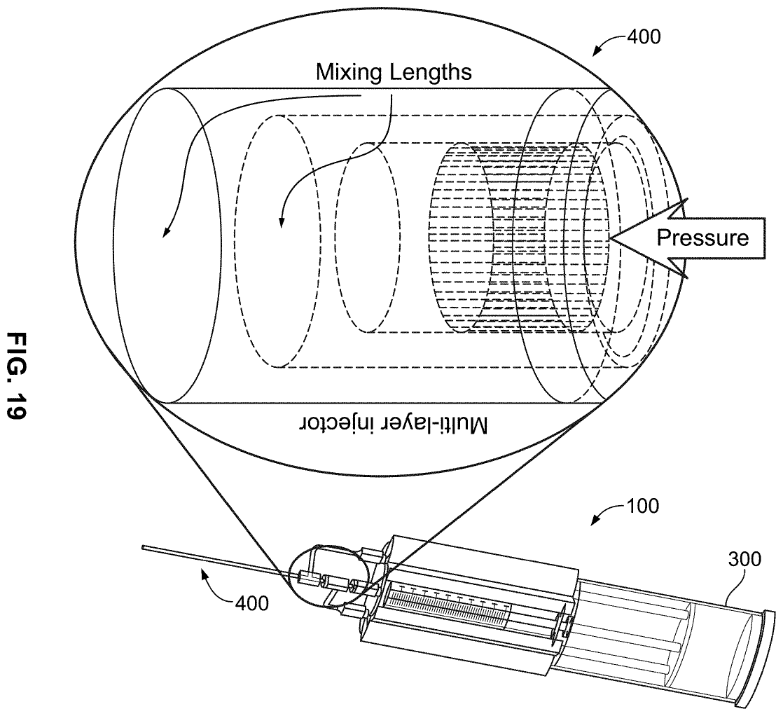

In the exemplary embodiment of FIGS. 17-20, like the printer device described above with reference to FIGS. 3-11, the printer device 100 similarly includes a syringe holder 200, a plunger 300, and an injector 400. However, in this embodiment, the syringe holder 200 is configured to hold three conventional syringes 50 containing material components of a three-part material intended to be delivered to a material deposition site. Accordingly, the injector 400 has similar structure to that described above, but is adapted to have multiple branched portions defining three separate dedicated conduits each terminating in a connector complementary to a connector on the syringes, e.g., a Luer-lock style connector, as best shown in FIGS. 17 and 18. The plunger similarly has similar structure to that described above, but is adapted to have multiple plunger portions for mating with the three separate syringes, as best shown in FIG. 19.

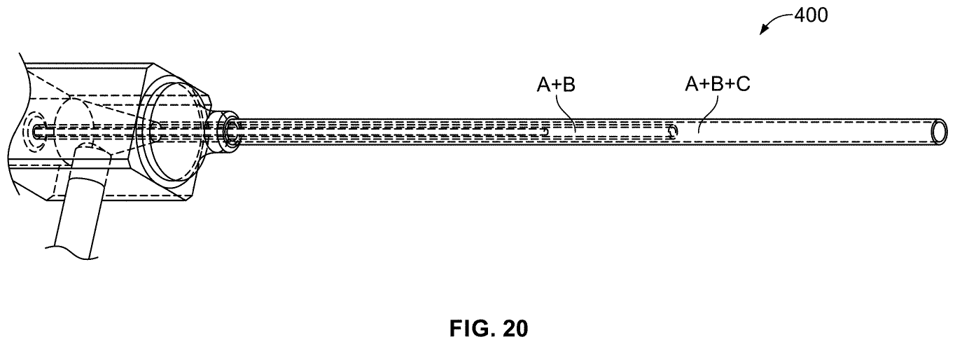

FIGS. 19 and 20 are partial views of the injector 400, which similarly defines coaxial passages that are not fully coextensive so three component materials A, B and C travel separately until they reach one or more common, mixing portions of the coextending portion of the injector 400. In this exemplary mixing portion, one or more material components (e.g., A and B) mix and flow together within the injector 400 in a first stage, subsequently, in a next stage of the mixing portion, the mixed A/B material may mix and flow together with material component C, to create a well-mixed A/B/C material. Accordingly, for cross-linkable materials, cross-linking may occur in the mixing region, before exiting at the distal end of the tip portion 450 of the injector 400, and before being deposited at a desired location. Again, the extent of cross-linking prior to can be controlled by varying the flow rate of the materials via the printer device, or by varying the structure of the injector. For example, the coextending portion 444 may be structured to have a longer or shorter mixing portion 470, in one or more stages, to cause greater or lesser mixing, and to provide greater or lesser dwell time allowing for cross-linking for a given flow rate, as may be desired for the materials to be used.

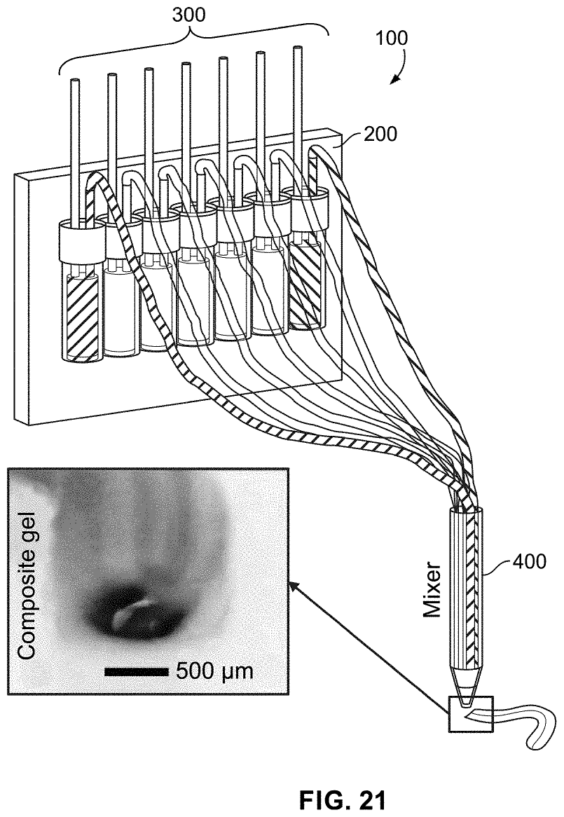

FIG. 21 illustrates another alternative embodiment of the printer device 100. In this embodiment, like the printer device described above with reference to FIGS. 3-11, the printer device 100 similarly includes a syringe holder 200, a plunger 300, and an injector 400. However, in this embodiment, the syringe holder 200 is configured to hold seven conventional syringes 50 containing material components of a seven-part material intended to be delivered to a material deposition site. Further, an external source of material component holders and external pressurized feeds to the handheld injector 400 are shown.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A printer for depositing material from syringes having a barrel and a plunger for dispensing material from the barrel via an outlet, said printer comprising:
a syringe holder defining at least two longitudinally-extending channels configured to receive and longitudinally constrain at least two syringes in predetermined spatial relationship, each syringe containing a respective component of said material;
a plunger defining at least two bosses for abutting at least two plungers of at least two syringes supported in said at least two longitudinally-extending channels; and
an injector comprising at least two connectors, each configured to connect to a respective one of said syringes, said injector defining at least two internal passages connectable to said syringes by said connectors, at least one of said internal passages terminating at a distal tip of said injector.

2. The printer of claim 1, wherein each of said at least two longitudinally-extending channels is dimensioned to receive one of a 1 mL syringe, a 3 mL syringe, a 5 mL syringe and a 10 mL syringe.

3. The printer of claim 2, wherein said at least two longitudinally-extending channels are dimensioned to receive syringes having different volumes.

4. The printer of claim 2, wherein said at least two longitudinally-extending channels are dimensioned to receive syringes having matching volumes.

5. The printer of claim 1, wherein said at least two longitudinally-extending channel extend along parallel axes.

6. The printer of claim 1, wherein said syringe holder defines at least one socket dimensioned for receiving a barrel flange of a syringe to longitudinally constrain said syringe.

7. The printer of claim 1, wherein said syringe holder defines a shoulder positioned to abut a portion of the barrel of a syringe to longitudinally constrain said syringe.

8. The printer of claim 7, wherein said shoulder is positioned to abut a barrel flange of the syringe.

9. The printer of claim 7, wherein said shoulder is positioned to abut a fitting of the syringe.

10. The printer of claim 1, wherein said at least two bosses of said plunger are connected by a common base to form an integral unit.

11. The printer of claim 1, wherein said syringe holder has a proximal end and a distal end, and wherein said syringe holder defines at least one through-bore extending and open to both its proximal end and its distal end.

12. The printer of claim 1, wherein said syringe holder has a proximal end and a distal end, and wherein said syringe holder defines at least two through-bores extending and open to both its proximal end and its distal end.

13. The printer of claim 1, wherein said injector has a proximal end and a distal end, and at least a portion of said internal passages extend coaxially within said injector.

14. The printer of claim 13, wherein said internal passages are not fully coextensive, said injector thereby defining a common mixing portion internally to said injector adjacent a distal end of one of said internal passages.

15. The printer of claim 13, wherein said internal passages are not fully coextensive, said injector thereby defining a common mixing portion internally to said injector adjacent a distal end of one of said internal passages, said mixing portion having a length configured to provide one of a desired mixing time and a desired cross-linking time for component materials carried by the syringes, for a given flow rate.

16. The printer of claim 1, wherein said injector has distal tip portion that is disposed at least 15 cm from a distal end of the syringe holder.

17. A printer for depositing material from syringes having a barrel and a plunger for dispensing material from the barrel via an outlet, said printer comprising:
a syringe holder defining at least two longitudinally-extending channels configured to receive and longitudinally constrain at least two syringes in predetermined spatial relationship, each syringe containing a respective component of said material;
a plunger defining at least two bosses for abutting at least two plungers of at least two syringes supported in said at least two longitudinally-extending channels;
an injector comprising at least two connectors, each configured to connect to a respective one of said syringes, said injector defining at least two internal passages connectable to said syringes by said connectors, at least one of said internal passages terminating at a distal tip of said injector; and
a leadscrew mechanism supported on said syringe holder and operable to mechanically drive said plunger with mechanical advantage.

18. The printer of claim 17, wherein said plunger defines threads, and wherein said leadscrew mechanism comprises:
a bevel lead screw having threads complementary to and mating with those of said plunger at one end, and beveled teeth at an opposite end;
a bevel gear having beveled teeth complementary to and mating with those of said bevel lead screw; and
a manually operated lever joined to said bevel lead gear, to cause rotation of said bevel gear in response to pivoting portion of said lever.

19. The printer of claim 18, further comprising a cover removably mountable to said syringe holder, said cover retaining said lever and bevel gear in meshing contact with the bevel lead screw when said cover is mounted to said syringe holder.

20. The printer of claim 19, further comprising a pin positioned inside said bevel gear, said pin transmitting torque from said lever to said bevel gear via a one-way needle bearing when moving against its free spinning direction.
